(19) United States
(12) Patent Application Publication
(10) Pub. No.: US 2010/0211094 A1
(43) Pub. Date: Aug. 19, 2010

(54) UMBRELLA DISTAL EMBOLIC PROTECTION DEVICE

(75) Inventor: Peter W. Sargent, JR., Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/372,987

(22) Filed: Feb. 18, 2009

(51) Int. Cl. A61F 2/01

(52) U.S. Cl. 606/200

(57) ABSTRACT

An embolic protection device for capturing emboli during treatment of a lesion in a blood vessel is presented. This embolic protection device generally comprises a core wire, a plurality of attachment cables and filter struts, and a filter member being configured to move between an expanded state for engagement with the blood vessel and a collapsed state for filter retrieval and delivery. The filter member is circumferentially attached to the attachment cables and filter struts and extends freely from its proximal end to a closed distal end. The core wire is rotated in a first direction to wrap the attachment cables, filter struts, and filter member around the core wire in the collapsed state and is rotated in a second or opposite direction to unwrap the attachment cables, filter struts, and filter member in the expanded state.

### FIELD

This invention relates generally to medical devices. More particularly, the present invention relates to embolic protection devices and methods for capturing emboli within a blood vessel.

### BACKGROUND

Due to the continuing advance of medical techniques, interventional procedures are becoming more commonly used to actively treat stenosis, occlusions, lesions, or other defects within a patient's body vessel. Often the region to be treated is located in a coronary, carotid, or cerebral artery, as well as in a peripheral vasculature or the kidneys. One example of a procedure for treating an occluded or stenosed body vessel is angioplasty. During angioplasty, an inflatable balloon is introduced into the occluded region. The balloon is inflated, pushing against the plaque or other material in the stenosed region. As the balloon presses against the material, portions of the material may inadvertently break free from the plaque deposit. These emboli may travel along the vessel and become trapped in smaller body vessels, which could result in restricting the blood flow to a vital organ, such as the brain.

To prevent the risk of damage from emboli, many devices have been used to restrict the flow of emboli downstream from a stenosed region. One such method includes inserting a balloon that may be expanded to occlude the flow of blood through the artery downstream of the stenosed region. An aspirating catheter positioned between the balloon and stenosed region may be used to remove any emboli resulting from the treatment. However, the use of this procedure is limited to very short intervals of time because the expanded balloon will completely block or occlude the blood flow through the vessel.

As an alternative to occluding flow through a blood vessel, various filtering devices have been used. Such devices typically have elements incorporating interlocking leg segments or a woven mesh that can capture embolic material, but allow blood cells to flow between the elements. Capturing the emboli in the filter device prevents the material from becoming lodged downstream in a smaller body vessel. The filter may subsequently be removed from the blood vessel along with the embolic material after the procedure has been performed and the risk from emboli has diminished.

However, various issues exist with the design, manufacturing, and use of existing filtering devices. Often it is desirable to deploy filter devices from the proximal side of the stenosed region. Therefore, the profile of the filtering device should be smaller than the opening through the stenosed region. In addition, the filter portion may become clogged or occluded during treatment, thereby, reducing the blood flow through the blood vessel. Moreover, many filtering devices are difficult to collapse and retrieve from the blood vessel after the need for such a device no longer exists.

Accordingly, there is a need to provide improved devices and methods for capturing emboli within a blood vessel, including providing distal protection during a procedure that has the potential to produce emboli without relatively restricting blood flow through the vessel and with the device being easily retrieved.

### SUMMARY

The present invention generally provides an embolic protection device used to collect emboli during the treatment of a stenotic lesion when deployed within the vasculature of a patient. The embolic protection device is relatively easy to deploy past the stenotic area and to be retrieved after the risk of releasing blood clots and thrombi within the vasculature has passed. The embolic protection device includes a core wire, a plurality of attachment cables and filter struts, and a filter member. The distal end of the attachment cables and the proximal end of the filter struts are coupled to the proximal end of the filter member. The proximal end of the attachment cables are coupled to the core wire, while the distal ends of the filter struts form a cage or basket structure. The distal end of the filter member is closed, thereby, forming an annular chamber useful for collecting emboli during treatment of the stenotic area. During treatment, the emboli are forced by the blood flow to move into the most distal part of the annulus chamber where it is caught or held.

The core wire, attachment cables, filter struts, and filter member are all one integral unit having a small cross sectional profile when the embolic protection device is in a coiled or collapsed state. Rotating the core wire in one direction causes the attachment cables, filter struts, and filter member to become wrapped around the core wire, thereby creating a small profile in the resulting collapsed state. Thus, during delivery of the device, this small profile enables the device to pass by a lesion without inadvertently dislodging material from the lesion site. After the device is distally located in reference to the stenotic area, rotating the core wire in the second or opposite direction results in the uncoiling or unwrapping of the attachment cables, filter struts, and filter member and the creation of an expanded state. Emboli formed during the subsequent treatment of the stenotic area will become trapped in the expanded filter member. The embolic protection device may then be retrieved by rotating the core wire to cause the attachment cables, filter struts, and filter member to become coiled or wrapped around the core wire, thereby, forming the collapsed state.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1A is a side-view of an embolic protection device in an uncoiled or expanded state in accordance with the teachings of the present invention;

FIG. 1B is a side-view of the embolic protection device of FIG. 2A in a coiled or collapsed state.

FIG. 2A is a sectional view of a blood vessel illustrating insertion of the embolic protection device of FIG. 1 B in its coiled or collapsed state;

FIG. 2B is a sectional view of a blood vessel illustrating the embolic protection device of FIG. 1A in its uncoiled or expanded state;

FIG. 2C is a sectional view of a blood vessel illustrating removal of the embolic protection device of FIGS. 2A and 2B from the vessel in its coiled or collapsed state;

FIG. 3A is a side view of an embolic protection assembly for capturing emboli during treatment in accordance with one embodiment of the present invention;

FIG. 3B is an exploded side view of the embolic protection assembly of FIG. 3A; and

FIG. 4 is a flow chart of one method for providing embolic protection during treatment of a stenotic lesion in a blood vessel according to the teachings of the present invention.

### DETAILED DESCRIPTION

The following description is merely exemplary in nature and is in no way intended to limit the present disclosure or its application or uses. It should be understood that throughout the description and drawings, corresponding reference numerals indicate like or corresponding parts and features.

The present invention generally provides an embolic protection device that is easy to deploy in a coiled or collapsed state within a vasculature of a patient. The embolic protection device in an uncoiled or expanded state effectively captures blood clots, thrombi, and other emboli resulting from the treatment of a lesion in the vasculature. In addition, the embolic protection device is relatively easy to retrieve after the risk associated with creating emboli in the vasculature during treatment has passed. One embodiment of the present invention generally provides an embolic protection device comprising a core wire; a plurality of filter struts, and attachment cables each having a proximal and distal end; and a filter member made of a polymer or cloth mesh membrane. The proximal end of the filter member is circumferentially attached to the proximal end of the filter struts and distal end of the attachment cables.

When deployed in a blood vessel, the attachment cables, filter struts, and filter member of the embolic protection device are uncoiled, thereby, allowing the filter struts and member to open into an expanded state that allows for blood to flow there through in order to capture emboli. The cables, struts, and filter member of the embolic protection device allow for relatively easy removal of the device from the blood vessel. This may be accomplished by coiling or wrapping the attachment cables, filter struts, and filter member around the core wire, thereby, creating the collapsed state for the device. The use of a sheath or catheter to assist in the deployment and retrieval of the embolic protection device is optional.

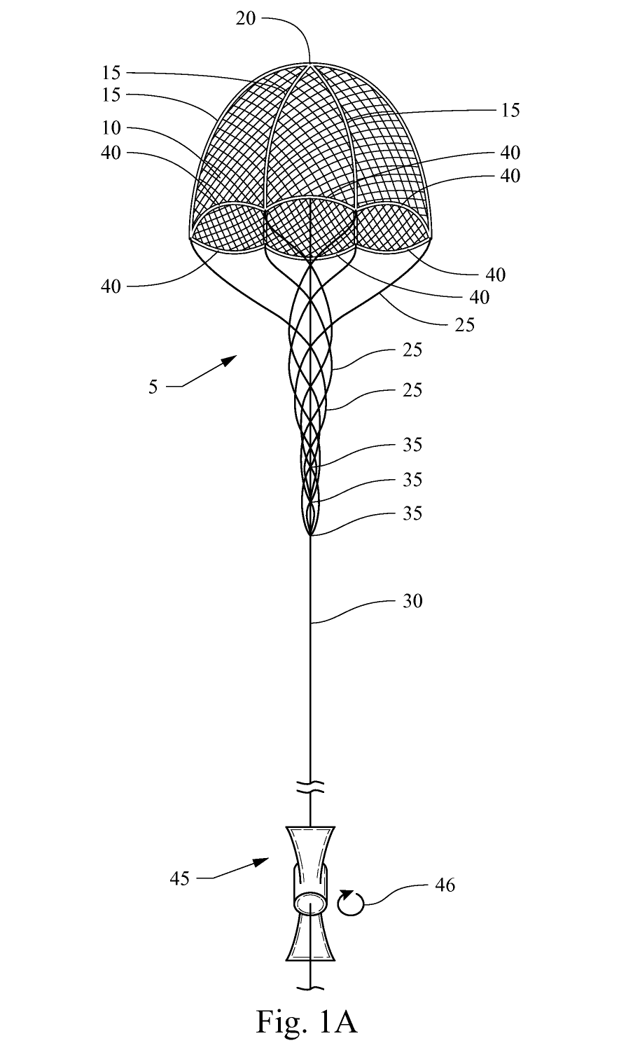

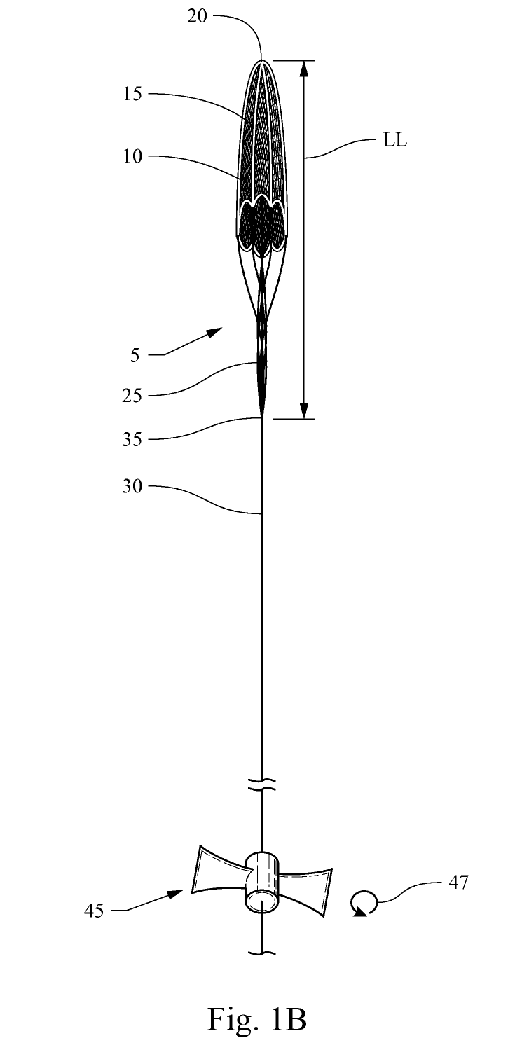

Referring to FIG. 1A, the embolic protection device 5 according to one embodiment of the present invention comprises a filter member 10 and a plurality of filter struts 15 each having a predetermined shape. Each filter strut 15 is attached to the filter member 10 in at least one location with multiple attachment locations along the length of the filter strut being desirable. The proximal ends of the filter struts 15 define the opening of a cage or basket structure to which the proximal end of the filter member is circumferentially attached. The distal end of the filter struts 15 are coupled together with the filter member 10 also being closed at its distal end. The distal end of the filter struts 15 may include a radiopaque tip 20 centrally aligned with the longitudinal axis, X, of the device.

The proximal end of the filter member 10 may also be coupled to more than one attachment cable 25. An attachment cable 25 is a flexible wire arranged such that it extends longitudinally from the core wire 30 at its proximal end to the opening of the cage or basket defined by the filter struts 15 and filter member 10 at its distal end. The proximal end of the attachment cables 25 may be coupled to the core wire 30 at attachment points 35. These attachment points 35 may be created using any biocompatible attachment mechanism known to one skilled-in-the-art, including but not limited to, glue and solder. Similarly, the distal end of the attachment cables 25 are coupled to the proximal end of the filter member 10 using a similar attachment mechanism. If desired, the points 40 of contact between the attachment cables 25 and the filter member 10 may be radiopaque. The distal end of the attachment cables 25 and the proximal end of the filter struts 15 can be coupled to the proximal end of the filter member 10 in different locations. However, if desired, an attachment cable 25 and a filter strut 15 may be coupled to the filter member 10 in the same or substantially similar location.

The core wire 30 of the embolic protection device 5 may be used as a guide wire for additional or other devices, such as a balloon catheter or stent catheter. The proximal end of the core wire 30 is coupled to an adjustable, rotatable wire clamp 45. During operation, the rotation of the wire clamp 45 in one direction 46 will cause the attachment cables 25 to unwrap or uncoil, thereby allowing the filter member 10 and filter struts 15 to also uncoil into an expanded state as shown in FIG. 1A. One skilled-in-the-art will recognize that the attachment cables 25 do not have to totally uncoil in order for the filter member 10 and filter struts 15 to become uncoiled to the extent necessary for the embolic protection device 5 to be fully deployed in its expanded state.

Referring now to FIG. 1 B, rotation of the wire clamp 45 in the opposite or second direction 47 will cause the filter member 10, filter struts 15, and attachment cables 25 to become wrapped or coiled around the core wire 30. As shown, the coiled or collapsed device 5 has a reduced diameter, occupying a cross-sectional profile less than the outer diameter of the device 5 in the expanded state (FIG. 1A). In this collapsed state, the embolic protection device may be either introduced into the vasculature of a patient or retrieved from said vasculature. The landing length of the embolic protection device 5 is defined as the longitudinal distance or length, LL, that extends between the attachment points 35 between the attachment cable 25 and core wire 30 on one end and the point at which the filter struts 15 are coupled together In order to create the closed basket structure on the other end (i.e., including the radiopaque tip 20). If desired, the core wire 30 may extend to and be coupled with the filter struts 15 at their distal end or with the radiopaque tip 20.

The filter member 10 extends freely from the attachment points 40 established between the distal end of the attachment cables 25 and the proximal end of the filter member 10 to its closed distal end, which is proximate to the radiopaque tip 20. The filter portion 10 forms at least one annulus chamber in its expanded state. During treatment, the emboli will be forced by the blood flow to move into the most distal part of the filter portion 10 where it is caught or held. Preferably, the longitudinal axis X of the embolic protection device 5 is positioned proximate to the center axis of the blood vessel.

The attachment cables 25 and filter struts 15 may be formed from materials, including but not limited to, a superelastic material, stainless steel, shape memory metal, cobalt-chromium-nickel-molybdenum-iron alloy, cobalt-chrome alloy, and Ni—Ti alloy (e.g., Nitinol). It is understood that the cables 25 or struts 15 may be formed of any suitable material known to one skilled-in-the-art that will result in a flexible structure. The attachment cables 25 and filter struts 15 may be made of the same or substantially similar material. However, it is preferable that the attachment cables 25 and filter struts 15 are constructed from different materials in order to allow them to exhibit different mechanical properties during use.

In one embodiment, the attachment cables 25 and filter struts 15 are made from Nitinol with a transition temperature that is slightly below normal body temperature of humans (that is, about 98.6° F.). Thus, when the embolic protection device 5 is fully deployed in a blood vessel and exposed to normal body temperature, the alloy of the cables 25 and struts 15 transforms from a martensite phase to an austenite phase (i.e., more rigid state). In order to remove the embolic protection device 5, the cables 25 and struts 15 may be wound or coiled around the core wire 30 when the adjustable clamp 45 is rotated.

The tip 20 and attachment points 40 may be made radiopaque by either the use of a noble metal or the application of a radiopaque polymeric or ceramic coating applied ay any suitable means, e.g., spraying or dipping. Examples of noble metals that may be used include gold, platinum, iridium, palladium, or rhodium, or a mixture thereof. The use of a radiopaque feature is suggested when it is desirable to provide a means to enhance fluoroscopy. The radiopaque feature of the tip 20 or attachment points 40 provides a means to more easily identify the embolic protection device during delivery, adjustment, or retrieval of the filter from the vasculature of the patient.

The filter member 10 may be formed from any suitable material for use in capturing emboli arising from a stenotic lesion during treatment without substantially reducing the flow of blood in the blood vessel. Preferably, the filter member 10 is made of a mesh/net cloth; nylon; polymeric material; poly(tetrafluoroethylene), such as Teflon® (DuPont de Nemours); or woven mixtures thereof. If desired, the filter member 10 may be pleated or folded.

In one embodiment, the filter portion 10 is made of a connective tissue material for capturing emboli. The connective tissue may include extracellular matrix (ECM), which is a complex structural entity surrounding and supporting cells that are found within mammalian tissues. The extracellular matrix can be made of small intestinal submucosa (SIS). SIS is a resorbable, acellular, naturally occurring tissue matrix composed of ECM proteins and various growth factors. SIS has characteristics of an ideal tissue engineered biomaterial and can act as a bioscaffold for remodeling of many body tissues including skin, body wall, musculoskeletal structure, urinary bladder, and also support new blood vessel growth.

In some implementations, SIS may be used to temporarily hold the filter member 10 against the walls of a blood vessel in which the device 5 is deployed. SIS has a natural affinity for body fluids and connective cells that form the connective tissue of a blood vessel wall. Because of the temporary nature of the duration in which the device 5 is deployed in the blood vessel, host cells of the wall will adhere to the filter member 10 but will not differentiate, allowing for retrieval of the device 5 from the blood vessel.

In use, the device 5 expands when unwrapped or uncoiled from its collapsed state to its expanded state. In an expanded state, the filter struts 15 will engage the wall of the blood vessel. In turn, the filter member 10 expands to capture emboli during treatment of the stenotic lesion. After the device 5 is no longer needed, it may be retrieved by wrapping or coiling the cables 25, struts 15, and filter member 10 around the core wire 30, thereby collapsing the device from its expanded state to its collapsed state. Optionally, a catheter may be deployed longitudinally about the embolic protection device 5 after it has been collapsed to assist in its retrieval.

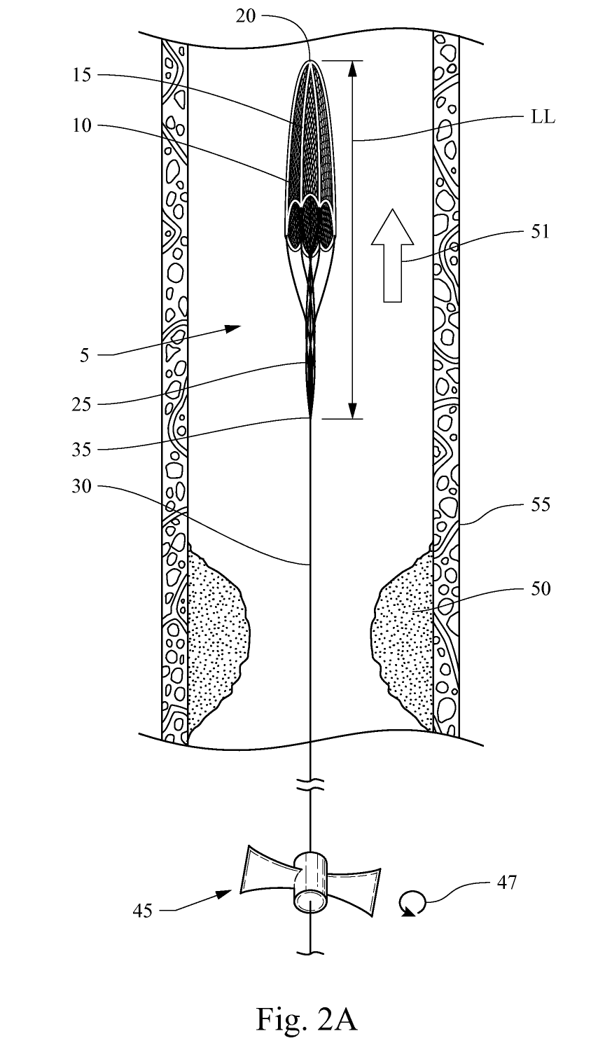

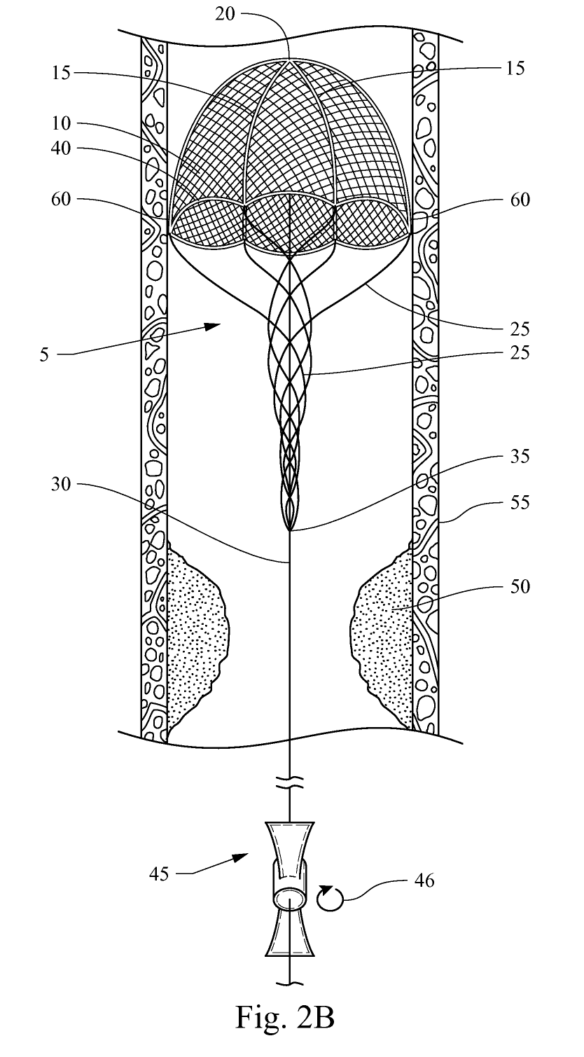

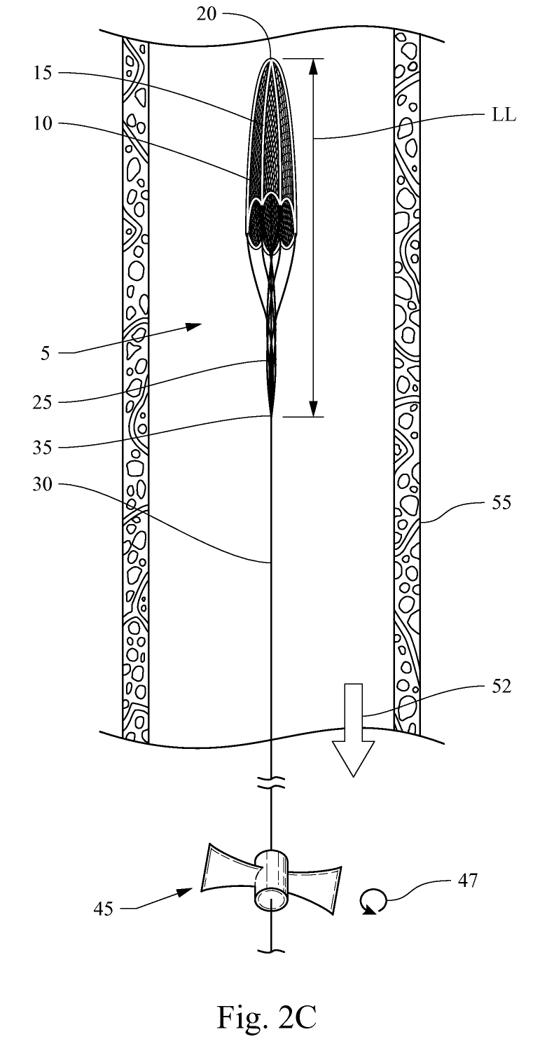

Now referring to FIG. 2A, a cutaway view of a blood vessel 55 is provided illustrating insertion of the embolic protection device 5. The embolic protection device 5 is inserted with the attachment cables 25, filter struts 15, and filter member 10 in a collapsed state, allowing the device 5 to navigate through the narrow opening that exists in the stenosed area 50. The device 5 is inserted past the stenosed area 50 by a distance that is at least equal to its landing length, LL. Accordingly, during insertion, the profile of the device 5 should be minimized. As such, the adjustable clamp 45 and the core wire 30 are rotated in one direction 46 causing the attachment cables 25, filter struts 15, and filter member 10 to wrap or become coiled around the core wire 30 forming a collapsed state. The small profile of the collapsed device enables the device 5 to pass by a stenosed lesion 50 without inadvertently dislodging material from the lesion site 50. The device 5 is inserted into the vessel 55 past the stenosis 50 as denoted by the distally pointing arrow 51.

Once the attachment cables 25, filter struts 15 and filter member 10 of the embolic protection device 5 are located distal to the stenosis 50, the cables 25, struts 15, and filter member 10 can be uncoiled and allowed to expand against the inner wall 60 of the blood vessel 55 as shown in FIG. 2B. In the expanded state, the filter struts 15 provide a radial force against the filter member 10, thereby securing the filter member 10 against the inner wall 60 of the vessel 55. The radial force eliminates gaps between the filter member 10 and the vessel 55 forcing embolic material that is released from the stenosis 50 to be trapped downstream in the annular chamber of the filter member 10. After a procedure is performed on the stenosis 50, the core wire 30 is rotated by turning the adjustable wire clamp 45 in one direction 47, thereby wrapping the attachment cables 25, filter struts 15, and filter member 10 around the core wire 30 creating the coiled or collapsed state as shown in FIG. 2C. In the collapsed state, the emboli are trapped within the annular chamber of the filter member 10 and against the core wire 30. Optionally, a catheter may also be slid over the device 5, as a precautionary measure during removal. The device 5 in the collapsed state may then be removed proximally, as denoted by the proximally pointing arrow 52.

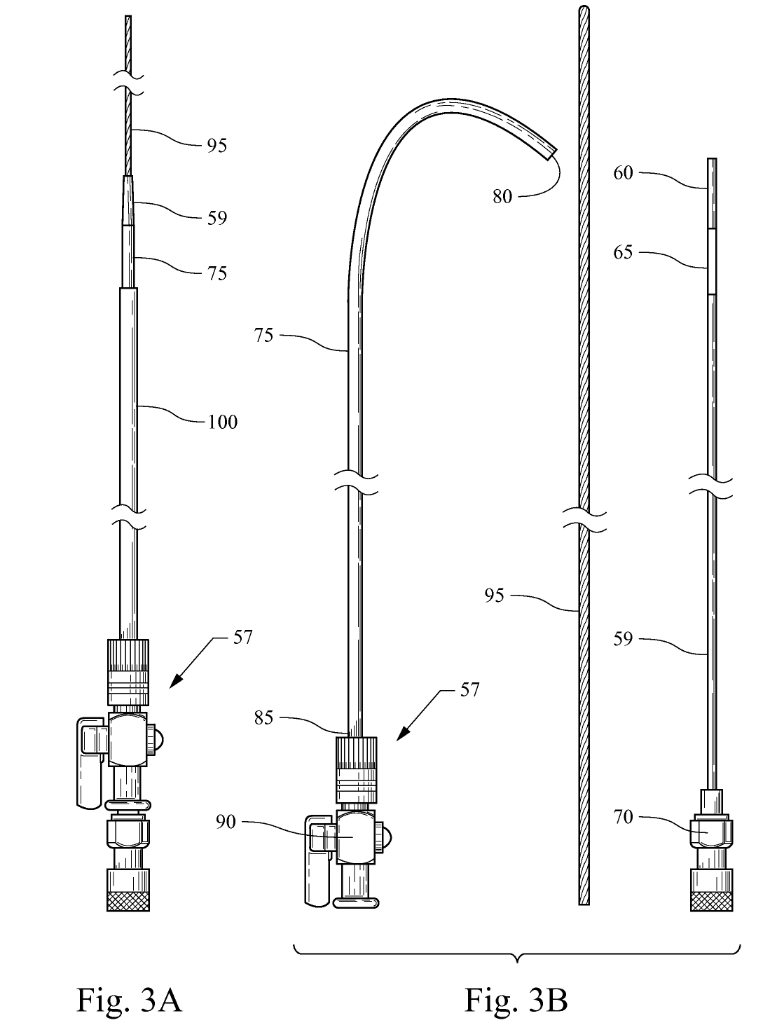

The embolic protection device 5 may be used independently without any other delivery system or mechanism. In fact, the device 5 may be used as the guide wire for deploying and retrieving other devices into the vasculature of a patient. Alternatively, the device 5 may be used, for example, with an embolic protection assembly 57as depicted in FIGS. 3A and 3B. As shown, the assembly 57 includes a balloon catheter 59 having a tubular body 62 and an expandable balloon 65 attached to and in communication with the tubular body 62 for angioplasty at a stenotic lesion. The assembly 57 also includes the embolic protection device 5 mentioned above. The tubular body 62 is preferably made of soft flexible material such as silicon or any other suitable material. The balloon catheter 59 may include an outer lumen that is in fluid communication with the balloon 65 for inflating and deflating the balloon 65 and an inner lumen formed within the outer lumen for percutaneous guidance through the blood vessel 55 with a wire guide and for deploying the embolic protection device 5. In certain implementations, the balloon catheter 59 has a proximal fluid hub 70 in fluid communication with the balloon 65 by way of the outer lumen for fluid to be passed through the outer lumen for inflation and deflation of the balloon 65 during treatment of the stenotic lesion.

The assembly 57 further includes an inner catheter 75 with a distal end 80 through which the balloon catheter 59 is disposed for deployment in the blood vessel 55. The inner catheter 75 is preferably made of a soft, flexible material such as silicon or any other suitable material. Generally, the inner catheter 75 also has a proximal end 85 and a plastic adaptor or hub 90 to receive the embolic protection device 5 and balloon catheter 59. The size of the inner catheter 75 is based on the size of the body vessel into which the catheter 75 is inserted, and the size of the balloon catheter 59.

The assembly 57 may also include a wire guide 95 configured to be percutaneously inserted within the vasculature to guide the inner catheter 75 to a location adjacent a stenotic lesion. Alternatively, the embolic protection device 5 with a core wire 30 may be employed as the wire guide 95 in the assembly 57.

To deploy the embolic protection device 5 according to one embodiment of the present invention, the device 5 is placed in the inner lumen of the balloon catheter 59 prior to treatment of the stenotic lesion. The distal protection device is then guided through the inner lumen preferably from the hub 70 and distally beyond the balloon 65 of the balloon catheter 59, exiting from the distal end of the balloon catheter 59 to a location within the vasculature downstream of the stenotic lesion where it can be uncoiled into the expanded state.

The assembly 57 may include a polytetrafluoroethylene (PTFE) introducer sheath 100 for percutaneously introducing the wire guide 95 and the inner catheter 75 in a blood vessel. Of course, any other suitable material known to one skilled-in-the-art may be used. The introducer sheath 100 may have any suitable size, e.g., between about three-french to eight-french. The introducer sheath 100 serves to allow the inner and balloon catheters 75, 65 to be inserted percutaneously to a desired location in the blood vessel. The introducer sheath 100 receives the inner catheter 75 and provides stability to the inner catheter at a desired location of the blood vessel. For example, as the introducer sheath 100 is held stationary within a common visceral artery, it adds stability to the inner catheter 75, as the inner catheter 75 is advanced through the introducer sheath 100 to a dilatation area in the vasculature.

When the distal end 80 of the inner catheter 75 is at a location downstream of the dilatation area in the blood vessel, the balloon catheter 59 is inserted through the inner catheter 75 to the dilatation area. The embolic protection device 5 is then loaded at the proximal end of the balloon catheter 59 and is advanced coaxially through the inner lumen of the balloon catheter 59 for deployment through the distal end of the balloon catheter 59. In this embodiment, the proximal end of the core wire 30 is used to mechanically advance the embolic protection device 5 through the catheter.

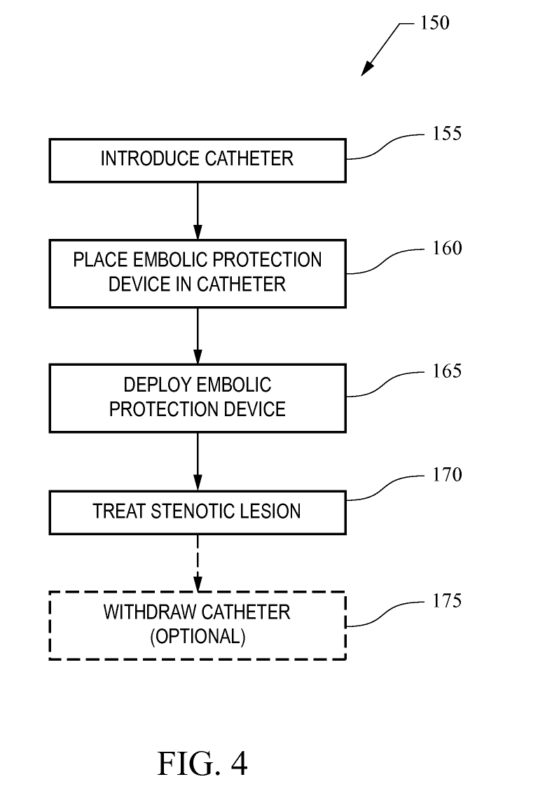

FIG. 4 depicts one method 150 for capturing emboli during treatment of a stenotic lesion in a body vessel 55, implementing the assembly 57 mentioned above. The method 150 comprises percutaneously introducing a balloon catheter 59 having an expandable balloon 65 for angioplasty of the stenotic lesion in the blood vessel 55 in step 155. Introduction of the balloon catheter 59 may be performed by any suitable means or mechanism. As mentioned above, an introducer sheath 100 and a wire guide 95 may be used to provide support and guidance to the balloon catheter 59. This wire guide 95 may be the embolic protection device 5 with core wire 30. For example, the wire guide 95 may be percutaneously inserted through the introducer sheath 100 to the stenotic lesion in the blood vessel 55. The inner catheter 75 and balloon catheter 59 may then be placed over the wire guide 95 for percutaneous guidance and introduction to the stenotic lesion 50. The physician may use any suitable means, for example, fluoroscopy, of verifying the placement of the balloon catheter 59 at a dilatation area.

The method 150 further comprises disposing the embolic protection device 5 coaxially within the balloon catheter 59 in step 160. The device 5 may be disposed coaxially within the balloon catheter 59 before or after percutaneous insertion of the balloon catheter 59. For example, once the balloon catheter 59 is placed at the stenotic lesion 50, the device 5 may then be disposed within the balloon catheter 59 for guidance and introduction in the body vessel 55. In this example, the expandable balloon 65 is positioned at the stenotic lesion 50 and the device 5, in its collapsed state, is disposed through the distal end of the balloon catheter 59 downstream from the expandable balloon 65.

The method 150 further includes deploying the device in a deployed or expanded state downstream from the stenotic lesion 50 to capture emboli during treatment of the stenotic lesion in step 165. In the expanded state, the open end of the filter portion 10 is expanded to a proximally facing concave shape for capturing emboli during angioplasty.

The method 150 may further include treating the stenotic lesion 50 in the blood vessel 55 with the balloon catheter 59 in step 170. In this step, the expandable balloon 65 may be injected with saline and expanded for predilatation. As desired, additional balloon catheters 59 may be used for pre-dilatation treatment, primary dilatation treatment, and post-dilatation treatment of the stenotic lesion while the device is in its expanded state within the blood vessel.

Finally, the method 150 may further comprise an optional step 175 in which the catheter is withdrawn. An alternative treatment device may then be placed if desired over the core wire 30 of the embolic protection device 5. In other words, the device 5 may serve as a wire guide for the delivery and retrieval of any alternative treatment devices.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An embolic protection device for capturing emboli during treatment of a stenotic lesion in a blood vessel, the device comprising:
a core wire;
a plurality of attachment cables having a proximal end and a distal end; the proximal end being coupled to the core wire;
a plurality of filter struts having a proximal end and a distal end; the distal end being joined together to forming a basket or cage structure; and
a filter member having a proximal end and a distal end with the filter member extending freely from the proximal end to a closed distal end forming at least one annulus chamber; the proximal end being circumferentially attached to the distal end of the attachment cables and the proximal end of the filter struts;
wherein the device is configured to move between an uncoiled state for engagement with the blood vessel and a coiled state for filter retrieval and delivery;
wherein the core wire is rotated in a first direction to wrap the attachment cables, filter struts, and filter member around the core wire in the coiled state and is rotated in a second or opposite direction to unwrap the attachment cables, filter struts, and filter member in the uncoiled state.

2. The embolic protection device of claim 1, wherein the device further comprises a radiopaque tip used to couple the distal ends of the filter struts in forming the basket or cage structure.

3. The embolic protection device of claim 1, wherein the annulus chamber of the filter member is configured to allow passage of blood through it and to capture emboli caused by the treatment of the stenotic lesion.

4. The embolic protection device of claim 1, wherein the filter member is made of one selected from the group of cloth, nylon, a polymeric material, poly(tetrafluoroethylene), extracellular matrix (ECM), small intestinal submucosa (SIS), and woven mixtures thereof.

5. The embolic protection device of claim 4, wherein the filter member is folded or pleated.

6. The embolic protection device of claim 1, wherein the attachment cables and filter struts are made of one selected from the group of a superelastic material, shape memory alloy, stainless steel wire, cobalt-chromium-nickel-molybdenum-iron alloy, cobalt-chrome alloy, nickel-titanium alloy, Nitinol, and mixtures thereof.

7. The embolic protection device of claim 6, wherein the attachment cables and the filter struts are constructed from a different material.

8. The embolic protection device of claim 1, wherein the attachment cables and the filter struts are attached to the filter member in different locations.

9. The embolic protection device of claim 1, wherein the proximal ends of the filter struts and filter member are configured to engage the blood vessel to anchor the device thereto.

10. A method for embolic protection during treatment of a stenotic lesion in a blood vessel, the method comprising the steps of:
introducing a catheter into the blood vessel;
placing the embolic protection device in the catheter in a collapsed state;
deploying an embolic protection device in a collapsed state into the blood vessel past the lesion and causing the device to move from the collapsed state to an expanded state in order to capture emboli during treatment, the device comprising:
a core wire;
a plurality of attachment cables having a proximal end and a distal end;
the proximal end being coupled to the core wire;
a plurality of filter struts having a proximal end and a distal end; the distal end being joined together to forming a basket or cage structure; and
a filter member having a proximal end and a distal end with the filter member extending freely from the proximal end to a closed distal end forming at least one annulus chamber; the proximal end being circumferentially attached to the distal end of the attachment cables and the proximal end of the filter struts; and
treating the stenotic lesion
wherein the core wire is rotated in a first direction to wrap the attachment cables, filter struts, and filter member around the core wire in the collapsed state and is rotated in a second or opposite direction to unwrap the attachment cables, filter struts, and filter member in the expanded state.

11. The method of claim 10, further comprising the step of withdrawing the catheter and using the core wire as a wire guide for the delivery of another treatment device into the blood vessel.

12. The method of claim 10, wherein during the step of deploying the embolic protection device moving from its collapsed state to the expanded state includes allowing the filter struts to engage the inner wall of the blood vessel, thereby, providing a radial force against the filter member that secures the filter member against the inner wall of the vessel.

13. An assembly for removing emboli from a body vessel during the treatment of a stenotic lesion, the assembly comprising:
an embolic protection device including a core wire, a plurality of attachment cables and filter struts, and a filter member being configured to move between an expanded state for engagement with the body vessel and a collapsed state for filter retrieval and delivery; the filter member circumferentially attached to the attachment cables and filter struts; the filter member extending freely from its proximal end to a closed distal end forming at least one annulus chamber in the expanded state; and
a balloon catheter having a tubular body portion and an expandable balloon attached to and in fluid communication with the tubular body portion; the balloon catheter facilitating delivery of the embolic protection device in the collapsed state to a position distal to the lesion in the body vessel;
wherein the embolic protection device is configured in the expanded state to allow blood to flow therethrough and to capture emboli in the annulus chambers of the filter portion.
wherein the core wire is rotated in a first direction to wrap the attachment cables, filter struts, and filter member around the core wire in the collapsed state and is rotated in a second or opposite direction to unwrap the attachment cables, filter struts, and filter member in the expanded state.

14. The assembly of claim 13 wherein the balloon catheter includes an outer lumen and an inner lumen, the outer lumen being in fluid communication with the balloon for inflating and deflating the balloon, the inner lumen being formed therethrough for percutaneous guidance through the body vessel.

15. The assembly of claim 13 further comprising:
an inner catheter having a distal end through which the balloon catheter is disposed for deployment in the body vessel; and
an introducer sheath through which the inner catheter is inserted for percutaneous insertion in the body vessel.

16. The assembly of claim 15, wherein the core wire acts as a wire guide configured to be disposed through the inner lumen of the balloon catheter for percutaneous guidance through the body vessel.

17. The assembly of claim 13, wherein the filter portion is made of one selected from the group of cloth, nylon, a polymeric material, poly(tetrafluoroethylene), extracellular matrix (ECM), small intestinal submucosa (SIS), and woven mixtures thereof, while the attachment cables and filter struts are made of one selected from the group of a superelastic material, shape memory alloy, stainless steel wire, cobalt-chromium-nickel-molybdenum-iron alloy, cobalt-chrome alloy, nickel-titanium alloy, and Nitinol.

18. The assembly of claim 17, wherein the attachment cables and filter struts are made from different materials.
